(19) United States
(12) Patent Application Publication
(10) Pub. No.: US 2026/0088154 A1
(43) Pub. Date: Mar. 26, 2026

(54) SYSTEMS AND METHODS FOR MEDICAL IMAGING PROTOCOL NAME STANDARDIZATION

(71) Applicant: GE Precision Healthcare LLC, Waukesha, WI (US)

(72) Inventors: Philippe Gerner, Strasbourg (FR); Mathieu Bedez, Ciknar (FR)

(21) Appl. No.: 18/896,079

(22) Filed: Sep. 25, 2024

(51) Int. Cl. G16H 30/20

(52) U.S. Cl. CPC G16H 30/20

(57) ABSTRACT

A system and method for medical imaging protocol name standardization includes generating a synthetic training dataset from medical standards in public documentation utilizing knowledge elicitation, wherein the synthetic training dataset includes, for a given language and a given imaging modality, a plurality of combinations of possible medical imaging protocol names for respective standard protocol codes of a plurality of standard protocol codes for each standard medical imaging protocol. The system and method also includes generating a lightweight text classification model from the synthetic training dataset utilizing machine learning. The system and method further includes utilizing the lightweight text classification model to receive a medical imaging protocol name and to output a list of most probable protocol codes based on the medical imaging protocol name.

### BACKGROUND

The subject matter disclosed herein relates to systems and methods for medical imaging protocol name standardization.

When handling data from imaging examinations (typically in digital imaging and communication in medicine (DICOM) form), protocol names are utilized to route an examination to a corresponding treatment. In particular, protocol names help technologists match clinical imaging protocols to orders. Only a specific order will match a single protocol. Protocols are precise instructions that define how a set of medical images should be acquired to maximize diagnostic quality, to deliver consistent scan quality, and to provide efficient and effective radiology service delivery. An example is dose monitoring, where radiation dose thresholds are given for some exam categories, and any examination has to be mapped to the right examination category via its hospital specific protocol name. In certain cases, this mapping is done entirely manually.

The biggest hurdle in handling medical imaging text is the amount and variety of abbreviations. Large language models (LLMs) are technically the best tools for handling this kind of text. But the best large language models available today that are both multilingual and medical imaging aware are not open source and they will be open source in the future. Medical imaging text, on the other hand contain protected health information (PHI). Thus, a healthcare product cannot send such data to a third party without very special contract terms to be agreed upon with a hospital/customer. Imaging scanners used protocols to scan patients. Many hospital organizations maintain their own sets of protocols to be utilized for specific scenarios and operations. However, these protocols need to be maintained for each scanner model as they are incompatible across vendors (e.g., original equipment manufacturers) and are often incompatible across the same scanner model family. Protocol compatibility can be defined as the ability to use the protocols from one scanner to do the scan in another scanner to achieve similar results without the need of manual modifications to the protocols. Individual modifications are not considered as it can depend on the preference of the person prescribing the scan. Creating a new protocol outside the scanner can be a challenge without having the scanner protocol management software in place. Likewise, driving a common outcome across the protocols is a challenge as it cannot be done outside the protocol management software. Thus, there is a need to cross transfer the protocols across various vendors to have consistency in the radiology department for which there is no solution presently in the field.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for medical imaging protocol name standardization is provided. The computer-implemented method includes generating, via a processing system comprising one or more processors, a synthetic training dataset from medical standards in public documentation utilizing knowledge elicitation, wherein the training dataset includes, for a given language and a given imaging modality, a plurality of combinations of possible medical imaging protocol names for respective standard protocol codes of a plurality of standard protocol codes for each standard medical imaging protocol. The computer-implemented method also includes generating, via the processing system, a lightweight text classification model from the synthetic training dataset utilizing machine learning. The computer-implemented method further includes utilizing, via the processing system, the lightweight text classification model to receive a medical imaging protocol name and to output a list of most probable protocol codes based on the medical imaging protocol name.

In another embodiment, a system is provided. The system includes a memory encoding processor-executable routines. The system also includes a processing system including one or more processors and configured to access the memory and to execute the processor-executable routines, wherein the process-executable routines, when executed by the processing system, cause the processing system to perform actions. The actions include generating a synthetic training dataset from medical standards in public documentation utilizing knowledge elicitation, wherein the synthetic training dataset includes, for a given language and a given imaging modality, a plurality of combinations of possible medical imaging protocol names for respective standard protocol codes of a plurality of standard protocol codes for each standard medical imaging protocol. The actions also include generating a lightweight text classification model from the synthetic training dataset utilizing machine learning. The actions further include utilizing the lightweight text classification model to receive a medical imaging protocol name and to output a list of most probable protocol codes based on the medical imaging protocol name.

In a further embodiment, a non-transitory computer-readable medium, the computer-readable medium including processor-executable code that when executed by a processing system including one or more processors, causes the processing system to perform actions. The actions include generating a synthetic training dataset from medical standards in public documentation utilizing knowledge elicitation, wherein the synthetic training dataset includes, for a given language and a given imaging modality, a plurality of combinations of possible medical imaging protocol names for respective standard protocol codes of a plurality of standard protocol codes for each standard medical imaging protocol. The actions also include generating a lightweight text classification model from the synthetic training dataset utilizing machine learning. The actions further include utilizing the lightweight text classification model to receive a medical imaging protocol name and to output a list of most probable protocol codes based on the medical imaging protocol name.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a schematic diagram of a system configured for medical imaging protocol name standardization, in accordance with aspects of the present disclosure;

FIG. 2 is a flow chart of a method for optimizing protocols for medical imaging protocol name standardization, in accordance with aspects of the present disclosure;

FIG. 3 is a flow chart of a method for generating synthetic training datasets, in accordance with aspects of the present disclosure;

FIG. 4 is a flow chart of a method for building a lightweight text classification model, in accordance with aspects of the present disclosure;

FIG. 5 is a flow chart of a method for utilizing a lightweight text classification model, in accordance with aspects of the present disclosure;

FIG. 6 is an example of a graphical user interface on a display showing generated training data, in accordance with aspects of the present disclosure;

FIG. 7 is a schematic diagram of a process for utilizing a lightweight text classification model (e.g., logistic regression model), in accordance with aspects of the present disclosure;

FIG. 8 is an example of a graphical user interface on a display showing generated n-grams (e.g., character shingles), in accordance with aspects of the present disclosure;

FIG. 9 is an example of a graphical user interface on a display for output of a lightweight text classification model (e.g., for Spanish), in accordance with aspects of the present disclosure; and

FIG. 10 is an example of a graphical user interface on a display for output of a lightweight text classification model (e.g., for French), in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers’ specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

The term processor, processing system, or processing unit, as used herein, refers to any type of processing unit that can carry out the required calculations needed for the various embodiments, such as single or multi-core: CPU, Accelerated Processing Unit (APU), Graphics Processing Unit, DSP, FPGA, ASIC or a combination thereof.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the terms “application”, “application module” (or “module”), “engine”, or “program”, or “plugin” refers to one or more sets of computer software instructions (e.g., computer programs and/or scripts) executable by one or more processors of a computing system to provide particular functionality. Computer software instructions can be written in any suitable programming languages, such as C, C++, C#, Fortran, Perl, MATLAB, SAS, SPSS, Python, JavaScript, and JAVA. Such computer software instructions can comprise an independent application with data input and data display aspects (e.g., modules). Alternatively, the disclosed computer software instructions can be classes that are instantiated as distributed objects. The disclosed computer software instructions can also be component software, for example JAVABEANS or ENTERPRISE JAVABEANS. Additionally, the disclosed applications or engines can be implemented in computer software, computer hardware, or a combination thereof.

As used herein, the terms “automatic” and “automatically” refer to actions that are performed by a computing device or computing system (e.g., of one or more computing devices) without human intervention. For example, automatically performed functions may be performed by computing devices or systems based solely on data stored on and/or received by the computing devices or systems despite the fact that no human users have prompted the computing devices or systems to perform such functions. As but one non-limiting example, the computing devices or systems may make decisions and/or initiate other functions based solely on the decisions made by the computing devices or systems, regardless of any other inputs relating to the decisions.

Deep learning (DL) approaches discussed herein may be based on artificial neural networks, and may therefore encompass one or more of deep neural networks, fully connected networks, convolutional neural networks (CNNs), transformer-based networks, unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides for systems and methods for medical protocol name standardization. In particular, the systems and methods enable mapping imaging protocol names in free text to standardized form, for the most common languages, with good tolerance to medical abbreviations. The disclosed systems and methods provide a short list of candidates for protocol standardization. Upon defining all of the mapping between medical standard and the examination categories, by transitivity, the solution can automatically provide a short list of which protocol names should map to which examination category.

The disclosed embodiments enable the best large language models to be utilized without any privacy issues. In addition, the disclosed embodiments support a large number of languages with limited human involvement as the needed medical knowledge (and associated variations in variation) is extracted from a large model with no privacy issues. The disclosed embodiments enable data to be generated in various languages from an English standard without explicit translation. The disclosed embodiments reduce the costs typically associate with manually mapping. The disclosed embodiments provide a classifier that is nearly as accurate as using a state of the art large language model (LLM). The disclosed embodiments also provide a much faster and cheaper option than using a large LLM as the classification models are comparatively light and have an inference time of milliseconds. The disclosed embodiments also enable the training set to be parameterized to needed cases (e.g., abbreviations).

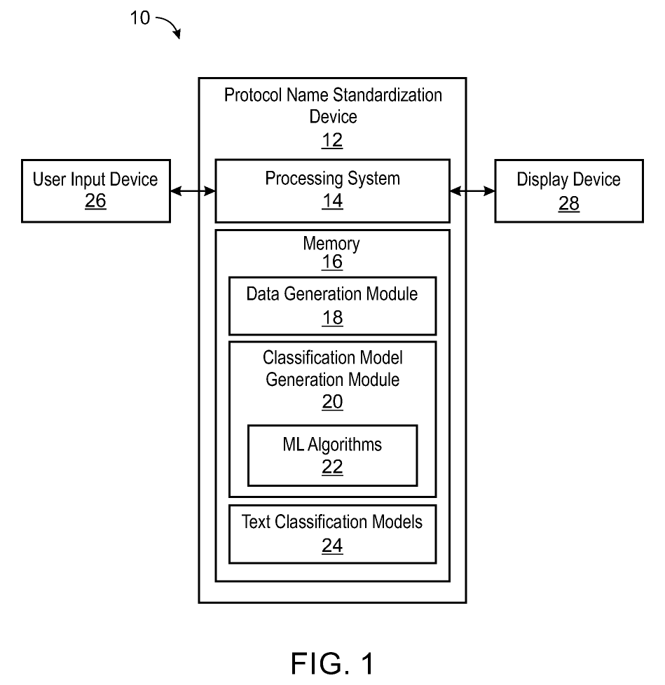

FIG. 1 is a schematic diagram of a system 10 (e.g., medical imaging protocol name standardization system) configured for medical imaging protocol name standardization (e.g., for scanning or radiological protocols for medical imaging scanners). A scanning protocol into the account the imaging modality, the purpose of the scan, the anatomical region of interest to be images, and scanning parameters (e.g., acquisition parameters). As depicted, the system 10 includes a protocol name standardization device 12 (e.g., implemented in a computing device). The protocol name standardization device 12 may be located on a medical imaging system or may located remotely from any medical imaging system. The protocol name standardization device 12 is configured to map medical imaging protocol names in free text to a standardized form for most common languages while providing good tolerance for medical abbreviations. The protocol name standardization device 12 is configured to generate synthetic data utilizing knowledge extraction. The protocol name standardization device 12 is also configured to utilize the generate synthetic data to generate lightweight text classification models (e.g., via standard machine learning algorithms). The protocol name standardization device 12 is further configured to utilize the lightweight text classification models to map the medical imaging protocol names to a standardized form.

The protocol name standardization device 12 includes one or more processors forming a processing system 14 configured to execute machine readable instructions stored in non-transitory memory 16. A processor of the processing system 14 may be single core or multi-core, and the programs executed thereon may be configured for parallel or distributed processing. In some embodiments, the processing system 14 may optionally include individual components that are distributed throughout two or more devices, which may be remotely located and/or configured for coordinated processing. In some embodiments, one or more aspects of the processing system 14 may be virtualized and executed by remotely-accessible networked computing devices configured in a cloud computing configuration.

The protocol name standardization device 12 also includes the non-transitory memory 16. The non-transitory memory 16 may store a data generation module 18. The data generation module 18 is configured to generate one or more synthetic training dataset from medical standards in public documentation (e.g., in the English language such as RadLex standard in radiology) utilizing knowledge elicitation. In particular, the data generation module may generate multiple datasets for a combination of a given language and a given imaging modality (e.g., computed tomography, magnetic resonance imaging, etc.). The training dataset, for a given language and a given imaging modality, includes a plurality of combinations of possible medical imaging protocol names for respective standard protocol codes of a plurality of standard protocol codes for each standard medical imaging protocol.

The data generation module 18 is configured to access and to utilize one or more LLMs. The LLMs provide radiology context and provide the benefit of attention (i.e., sequences of words/abbreviations having meaning together). In certain embodiments, the LLMs are closed-source (e.g., GPT-3.5, GPT-4, and GPT-4o). In certain embodiments, the LLMs are open-source (e.g., Llama family and others). In certain embodiments, the LLMs are medical language specific. The data generation module 18 is configured to receive one or more prompts or user inputs in utilizing the one or more LLMs. The prompts may include a target language, a target modality, a target number of protocol name variations to generate, and/or other information. In certain embodiments, R may be utilized for scripting when utilizing the LLMs. The data generation module 18 is configured to obtain a respective block of information pertaining to each standard protocol code of the plurality of standard protocol codes from the public documentation, wherein the public documentation is in the English language. The data generation module 18 is configured to, for each respective block of information for each standard protocol code, generate with a LLM (via prompting via user input), an expanded and explicit text description (i.e., whole protocol description) in the English language for the standard medical imaging protocol. In prompting the LLM, variation may be requested as well as frequent abbreviations. Also, inference parameters (e.g., temperature and/or repetition avoidance parameters) of the LLM may be adjusted in generating the synthetic training dataset. The data generation module 18 is configured to, from each expanded and explicit text description, generate with a multilingual medical large language model (LMM) a set of medical imaging protocol names (whole protocol names) in the given language with multiple variations and multiple abbreviations. In certain embodiments, the given language is also the English language. In certain embodiments, the given language is different from the English language. The data generation module 18 is configured to generate multiple sets of medical imaging protocol names in the given language with multiple variations and multiple abbreviations. The data generation module 18 is configured to attach each medical imaging protocol name of the set of imaging protocol names to a corresponding standard protocol code from which it originated to define source-target lines of the synthetic training dataset for the given language. In certain embodiments, the synthetic training dataset is generated for multiple different languages for the given imaging modality. In certain embodiments, the synthetic training dataset is generated for multiple different languages for multiple different imaging modalities. Since the chosen medical standard is public (by definition) and PHI-free, there is no infringement of privacy in the generation of the training dataset.

In the process utilized by the data generation module 18, the medical abbreviations are always part of/embedded in a very signifying context. Thus, the process ensures that the abbreviations are generated based on a (expressly generated) extended description of the protocol instead of starting from some words to abbreviate. Thus, even abbreviations that were not necessarily in the LLM training set can be generated. Also, the data is generated by modality so that each imaging modality has its own set of medical imaging notions. In addition, the training dataset can be made as large and as well balanced as desired. Balance meaning that each class is equally well represented in the training dataset. Both the size and balance of the dataset contributes to the accuracy of the predictions of the classification models generated with the dataset.

The non-transitory memory 16 may also store a classification model generation module 20. The non-transitory memory 16 may also store one or more machine learning algorithms 22 (e.g., standard machine learning algorithms). The classification model generation module 20 is configured to generate one or more lightweight text classification models 24 from one or more synthetic training datasets utilizing the machine learning (ML) algorithms 22. In certain embodiments, Python may be utilized for model building or generation. A lightweight text classification model reduces computational complexity, memory usage, and power consumption compared to a large LLM. The classification model generation module 20 is configured to generate respective lightweight text classification models 24 for different combinations of languages and imaging modalities from the synthetic training dataset utilizing machine learning (i.e., a lightweight text classification model for a combination of a single imaging modality and a single language). Each lightweight text classification model 24 is configured to receive a medical imaging protocol name (e.g., in a given or target language) and to output a list of most probable protocol codes based on the medical imaging protocol name. Besides the most probable protocol codes, the lightweight text classification model 24 may also output a shore name for each probable protocol code. In certain embodiments, the lightweight text classification model 24 is configured to utilize logistic regression in determining the list of most probable protocol codes based on the medical imaging protocol name. In certain embodiments, the lightweight text classification model 24 is configured to determine a most probable protocol based on the medical imaging protocol name. In certain embodiments, the lightweight text classification model is configured to calculate and output a respective confidence score for each protocol code in the list of most probable protocol codes. The confidence score may be a numerical score or a number of symbols (e.g., stars). In certain embodiments, the language of the medical imaging protocol name that is received is detected. Based on the detected language, a corresponding lightweight text classification model 24 from the respective lightweight text classification models 24 is selected to utilize.

In some embodiments, non-transitory memory 16 may include components disposed at two or more devices, which may be remotely located and/or configured for coordinated processing. In some embodiments, one or more aspects of non-transitory memory 16 may include remotely-accessible networked storage devices configured in a cloud computing configuration.

User input device 26 may include one or more of a touchscreen, a keyboard, a mouse, a trackpad, a motion sensing camera, or other device configured to enable a user to interact with the protocol name standardization device 12. In one example, user input device 26 may enable a user to input a medical imaging protocol name into a lightweight text classification model 24. In another example, user input device 26 may enable a user to input prompts data generation module 18. These prompts may include a target language, a target imaging modality, a target number of protocol name variations to generate, and/or other information (e.g., desired variability via different parameter values, adding a self-curation step, etc.). Prompts may also relate on how input text is to be analyzed/expanded. Prompts may also ask for a few typos (e.g., as they occur in real protocol names). Prompts may relate to adding more precise instructions. In certain embodiments, the prompting is zero-shot prompting. In certain embodiments, the prompting is few-shot prompting.

Display device 28 may include one or more display devices utilizing virtually any type of technology. In some embodiments, the display device 28 may include a computer monitor, and may display the most probable protocol codes, associated short names, and confidence scores. Display device 28 may be combined with the processing system 14, the non-transitory memory 16, and/or the user input device 26 in a shared enclosure, or may be peripheral display devices and may comprise a monitor, touchscreen, projector, or other display device known in the art, which may enable a user to view data and/or interact with various data stored in the non-transitory memory 16.

The processing system 14 is configured to generate a synthetic training dataset from medical standards in public documentation utilizing knowledge elicitation (e.g., automatic knowledge elicitation), wherein the synthetic training dataset includes, for a given language and a given imaging modality, a plurality of combinations of possible medical imaging protocol names for respective standard protocol codes of a plurality of standard protocol codes for each standard medical imaging protocol. The processing system 14 is also configured to generate a lightweight text classification model from the synthetic training dataset utilizing machine learning. The processing system 14 is further configured to utilize the lightweight text classification model to receive a medical imaging protocol name and to output a list of most probable protocol codes based on the medical imaging protocol name.

The processing system 14 is configured to obtain a respective block of information pertaining to each standard protocol code of the plurality of standard protocol codes from the public documentation, wherein the public documentation is in the English language. The processing system 14 is also configured to generate the synthetic training dataset by generating, for each respective block of information for each standard protocol code, a large language model, an expanded and explicit text description in the English language for the standard medical imaging protocol. The processing system 14 is also configured to generate the synthetic training dataset by, from each expanded and explicit text description, generating with a multilingual medical large model a set of medical imaging protocol names in the given language with multiple variations and multiple abbreviations. The processing system 14 is also configured to generate the synthetic training dataset by generating multiple sets of medical imaging protocol names in the given language with multiple variations and multiple abbreviations. The processing system 14 is also configured to generate the synthetic training dataset by attaching each medical imaging protocol name of the set of imaging protocol names to a corresponding standard protocol code from which it originated to define source-target lines of the synthetic training dataset for the given language. The processing system 14 is also configured to generate respective lightweight text classification models for different combinations of languages and imaging modalities from the synthetic training dataset utilizing machine learning; detecting a language of the medical imaging protocol name that is received; and selecting a corresponding lightweight text classification model from the respective lightweight text classification models to utilize based on the language that is detected.

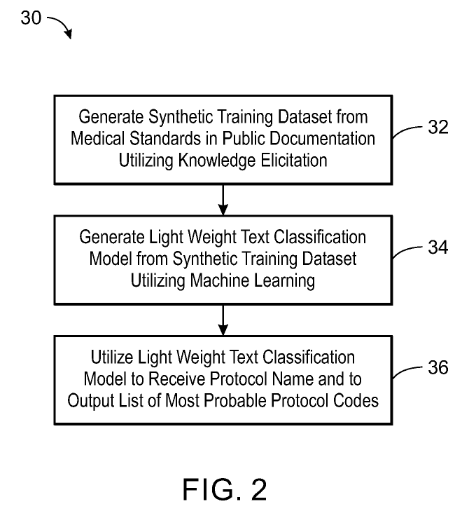

FIG. 2 is a flow chart of a method for optimizing protocols for medical imaging protocol name standardization. One or more steps of the method 30 may be performed by one or more components of the protocol name standardization device 12 in FIG. 1.

The method 30 includes generating a synthetic training dataset from medical standards in public documentation (e.g., in the English language such as RadLex standard in radiology) utilizing knowledge elicitation (block 32). The synthetic training dataset includes, for a given language and a given imaging modality, a plurality of combinations of possible medical imaging protocol names for respective standard protocol codes of a plurality of standard protocol codes for each standard medical imaging protocol. In certain embodiments, multiple synthetic training datasets may be generated for the same combination of a given language and a given imaging modality. In certain embodiments, one or more training datasets for different combinations of different languages and different imaging modalities. In certain embodiments, the given language may be the English language. In certain embodiments, the given language may a different language from the English language.

The method 30 also includes generating a lightweight text classification model from the synthetic training dataset utilizing machine learning (block 34). In certain embodiments, the lightweight text classification model may be built to predict a number of labels (e.g., up to 1300 for RadLex CT). In certain embodiments, the lightweight text classification model may be built for multiple languages. The collective aspect of the dataset automatically mitigates for hallucinations if some were to occur sometimes in the training dataset. In certain embodiments, generation of the lightweight text classification model includes fine-tuning a small LLM. In certain embodiment, standard machine learning algorithms may be utilized in building the model (e.g., logistic regression).

The method 30 further includes utilizing the lightweight text classification model to receive a medical imaging protocol name and to output a list of most probable protocol codes based on the medical imaging protocol name (block 36). A lightweight text classification model reduces computational complexity, memory usage, and power consumption compared to a large LLM. The lightweight text classification model is configured to receive a medical imaging protocol name (e.g., in a given or target language) and to output a list of most probable protocol codes based on the medical imaging protocol name. Besides the most probable protocol codes, the lightweight text classification model may also output a shore name for each probable protocol code. In certain embodiments, the lightweight text classification model is configured to utilize logistic regression in determining the list of most probable protocol codes based on the medical imaging protocol name. In certain embodiments, the lightweight text classification model is configured to determine a most probable protocol based on the medical imaging protocol name. In certain embodiments, the lightweight text classification model is configured to calculate and output a respective confidence score for each protocol code in the list of most probable protocol codes. The confidence score may be a numerical score or a number of symbols (e.g., stars).

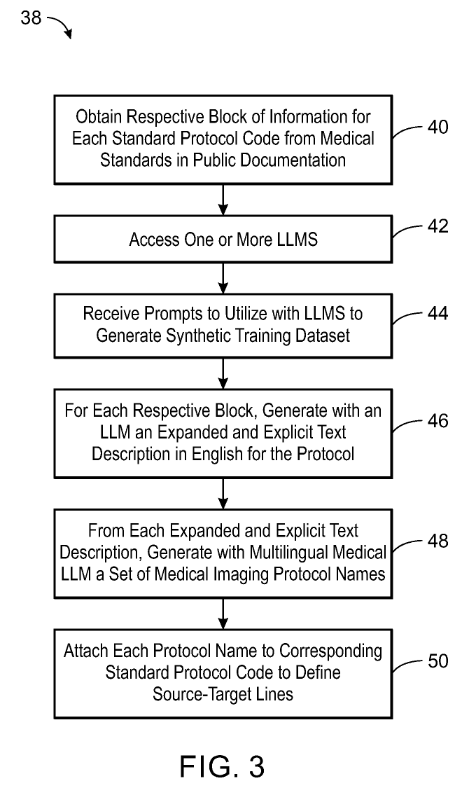

FIG. 3 is a flow chart of a method 38 for generating synthetic training datasets. One or more steps of the method 38 may be performed by one or more components of the protocol name standardization device 12 in FIG. 1.

The method 38 includes obtaining a respective block of information pertaining to each standard protocol code of a plurality of standard protocol codes from medical standards in public documentation (e.g., RadLex), wherein the public documentation is in the English language (block 40). The method 38 includes accessing one or more LLMs (block 42). The LLMs provide radiology context and provide the benefit of attention (i.e., sequences of words/abbreviations having meaning together). In certain embodiments, the LLMs are closed-source (e.g., GPT-3.5, GPT-4, and GPT-4o). In certain embodiments, the LLMs are open-source (e.g., Llama family and others). In certain embodiments, the LLMs are medical language specific.

The method 38 includes receiving one or more prompts (e.g., user inputs) for utilizing the one or more LLMs in generating a synthetic training dataset (block 44). The prompts may include a target language, a target imaging modality, a target number of protocol name variations to generate, and/or other information (e.g., desired variability via different parameter values, adding a self-curation step, etc.). Prompts may also relate on how input text is to be analyzed/expanded. Prompts may also ask for a few typos (e.g., as they occur in real protocol names). Prompts may relate to adding more precise instructions. In certain embodiments, the prompting is zero-shot prompting. In certain embodiments, the prompting is few-shot prompting.

The method 38 also includes, for each respective block of information for each standard protocol code (e.g., for a given or target imaging modality), generating (e.g., inferring) with an LLM, an expanded and explicit text description (e.g., whole text description) in the English language for the standard medical imaging protocol (block 46). The method 38 further includes, from each expanded and explicit text description, generating with a multilingual medical LLM a set of medical imaging protocol names in a given or target language with multiple variations and multiple abbreviations for a given imaging modality (block 48). In certain embodiments, the given language may be the English language. In certain embodiments, the given language may a different language from the English language. The method 38 even further includes attaching each medical imaging protocol name of the set of imaging protocol names to a corresponding standard protocol code from which it originated to define source-target lines of the synthetic training dataset for the given language (block 50). In certain embodiments, blocks 46-50 of the method 38 may be repeated more than once to generating multiple sets of medical imaging protocol names in the given language with multiple variations and multiple abbreviations. In certain embodiments, blocks 46-50 of the method 38 may be conducted for multiple different languages for the given image modality. In certain embodiments, blocks 46 and 48 of the method 38 may be performed simultaneously.

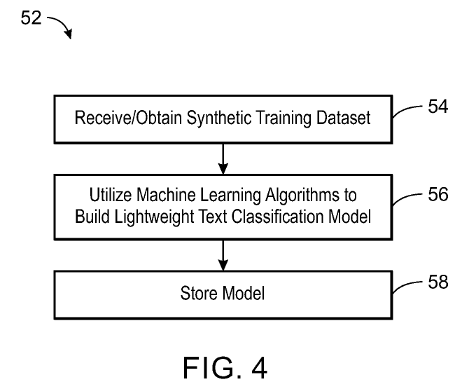

FIG. 4 is a flow chart of a method 52 for building a lightweight text classification model. One or more steps of the method 52 may be performed by one or more components of the protocol name standardization device 12 in FIG. 1.

The method 52 includes receiving or obtaining the one or more synthetic training datasets (as generated in the method 38 in FIG. 2) (block 54). The method 52 also includes utilizing one or more machine learning algorithms (e.g., standard machine learning algorithms) to build or generate a lightweight text classification model from one or more synthetic training datasets (block 56). An example of a machine learning algorithm utilized to build a lightweight text classification model is logistic regression. In certain embodiments, the lightweight text classification model may be built for a single given language and a given imaging modality. In certain embodiments, the lightweight text classification model may be built for multiple different languages for a given imaging modality. The collective aspect of the dataset automatically mitigates for hallucinations if some were to occur sometimes in the training dataset. In certain embodiments, generation of the lightweight text classification model includes fine-tuning a small LLM. The method 52 further includes storing the lightweight text classification model (e.g., on the protocol name standardization device 12 in FIG. 1) (block 58). The method 52 may be utilized to generate multiple text classification models for different combinations of languages and imaging modalities (i.e., one or more languages associated with a different respective imaging modality for each model).

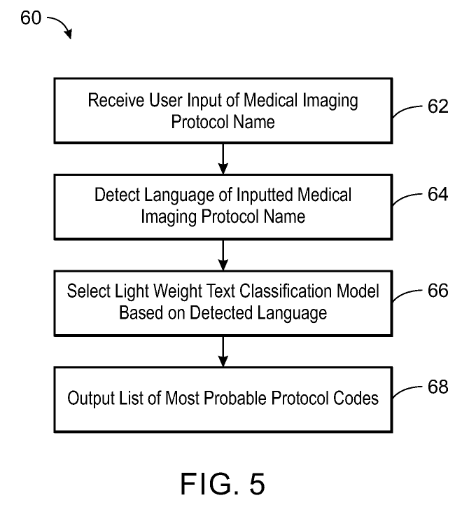

FIG. 5 is a flow chart of a method 60 for utilizing a lightweight text classification model. One or more steps of the method 60 may be performed by one or more components of the protocol name standardization device 12 in FIG. 1.

The method 60 includes receiving a user input of a medical imaging protocol name (block 62). The inputted medical imaging protocol name may have abbreviations and/or typos. In certain embodiments, the method 60 includes detecting a language of the medical imaging protocol name that is received or inputted (block 64). In certain embodiments, the method 60 also includes selecting (if there are multiple models) a corresponding lightweight text classification model from a plurality of lightweight text classification models to utilize based on the language that is detected in the medical imaging protocol name (block 66). The method 60 further includes outputting from the lightweight text classification model (e.g., selected lightweight text classification model) a list of most probable protocol codes based on the medical imaging protocol name (e.g. for a given medical imaging modality) (block 68). The list of most probable protocol codes may also be accompanied by short name for the respective protocol code in English. In certain embodiments, the lightweight text classification model calculates and outputs a respective confidence score for each protocol code in the list of most probable protocol codes. The confidence score may be a numerical score or a number of symbols (e.g., stars). In certain embodiments, prior to outputting the confidence score, a conformal regression may be applied to the initial or raw confidence score calibration purposes. In certain embodiments, the lightweight text classification model determines a most probable protocol based on the medical imaging protocol name. The confidence scores provides trust in the outcome of the machine learning-based text classification models (which is not possible if a LLM was utilized to due classification do to hallucination problem). In certain embodiments, through more prompting, lightweight text classification model may self-access its own confidence (in case the lightweight text classification model hallucinates its own confidence estimation).

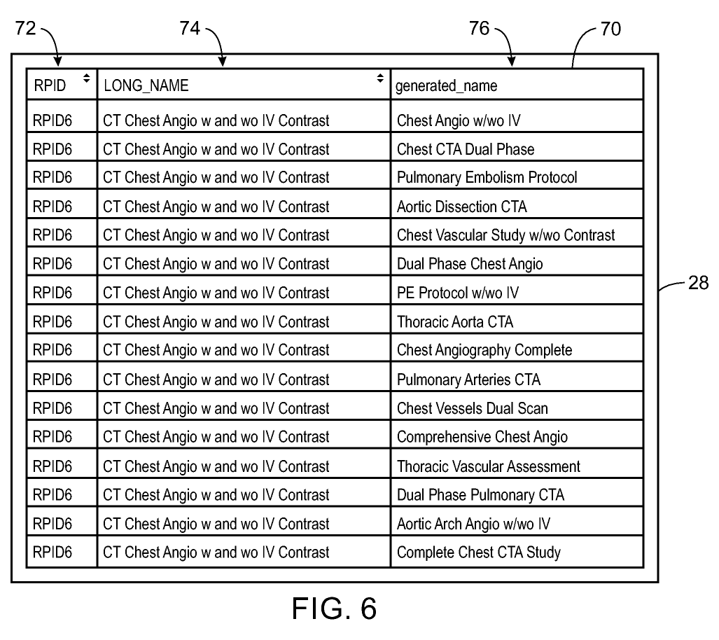

FIG. 6 is an example of a graphical user interface 70 on a display 28 showing generated training data (e.g., synthesized training data). The training data was generated utilizing the data generation module 18 in FIG. 1. Knowledge elicitation was utilized to generate the training data. The target imaging modality was computed tomography. The medical standards in public documentation is the RadLex standard in radiology. For a standard medical imaging protocol, an LLM was prompted to generate an expanded and explicit text description (e.g., whole description) for each protocol code based on its description in the public documentation (i.e., RadLex). Column 72 are the protocol codes (i.e., RPID) for the protocol. Column 74 are these expanded and explicit text descriptions (i.e., LONG_NAME) for each protocol code (which are more informative than the codes). An LLM (e.g., multilingual medical LLM) was also prompted from this expanded and explicit description to list protocol name variations with multiple variations and multiple abbreviations. Both the prompt and inference parameters (e.g., temperature) asked for frequent variation and frequent abbreviations. Column 76 are the generated protocol name variations (i.e., generated_name). Since the chosen medical standard is public (by definition) and PHI-free, there is no infringement of privacy in the generation of the training dataset. This process may be repeated multiple times in order to aggregate lists of many variations.

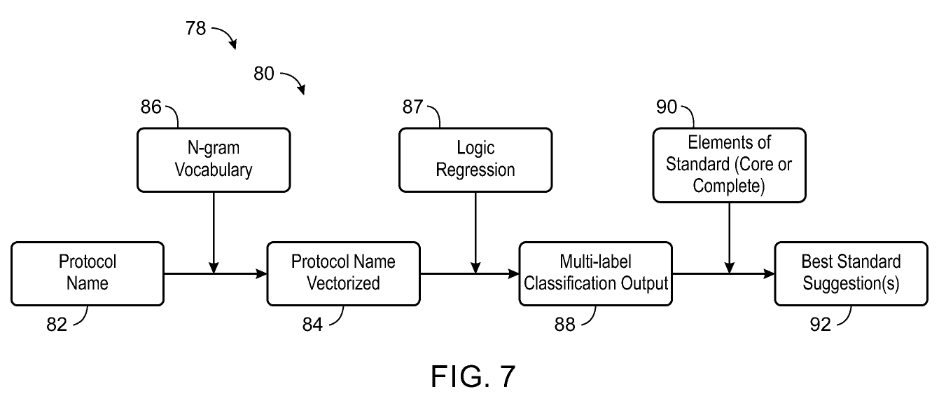

FIG. 7 is a schematic diagram of a process 78 for utilizing a lightweight text classification model 80 (e.g., logistic regression model). The lightweight text classification model 80 was built utilizing one or more standard machine learning algorithms (in particular, logistic regression). As depicted, the process 78 includes inputting a medical imaging protocol name 82 (i.e., protocol name strings) into the lightweight text classification model 80. The process 78 includes vectorizing or discretizing the inputted medical imaging protocol name 82 into a vectorized protocol name 84 with an N-gram vocabulary 86 (i.e., sequence of given number of adjacent letters in a specific order). This provides a robustness to the variety of abbreviations. A variety of different type of N-grams may be utilized. For example, character shingles may be utilized. The process 78 also includes utilizing logistic regression 87 on the vectorized protocol name 84 to perform and to output a multi-label classification 88. In certain embodiments, other standard machine learning algorithms may be utilized (e.g., support vector machines, naive Bayes, etc.). The process 78 includes applying elements of standard 90 (e.g., core or complete) to the multi-label classification output 88 to output the best suggestions from the standard 92 (i.e., list of most probable protocol codes based on the medical imaging protocol name). In certain embodiments, the list of most probable protocol codes may be accompanied with respective confidence scores based on the probability of the predictions (in conjunction with conformal prediction). In certain embodiments, the most probable protocol code is outputted. In certain embodiments, there may be multiple small or lightweight text classification models. In this scenario, combinations of imaging modality and languages are handled by a mixture of experts of approach (i.e., a small or lightweight text classification model is built for each combination).

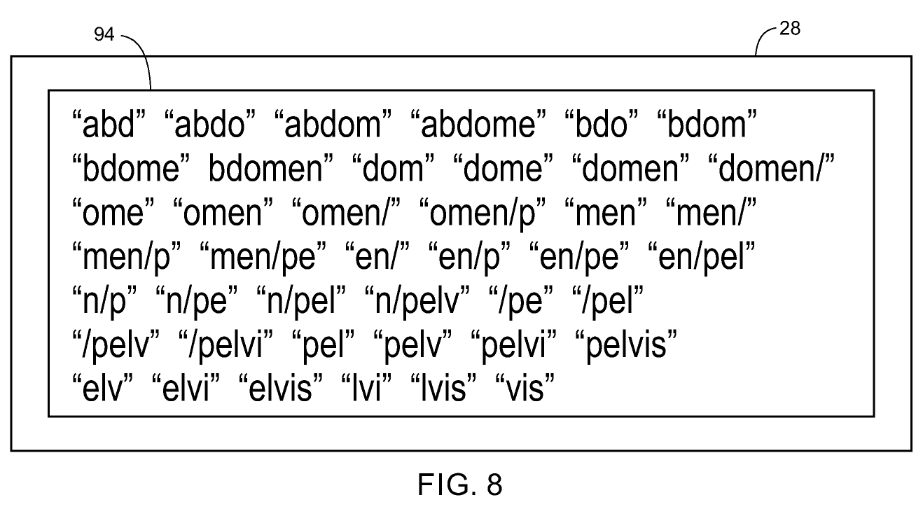

FIG. 8 is an example of a graphical user interface 94 on a display 28 showing generated n-grams. The following n-grams were generated utilizing character shingles. In the example in FIG. 8, “abdomen/pelvis” was generated (e.g., by an LLM) as an example for code (C). Shingles of 3 to 6 letters were chosen. In certain embodiments, other choices are possible. The graphical user interface 94 depicts the list generated with the character shingles. Each of these small sequence of letters acts as a signature for abdomen/pelvis and, thus, for code (C).

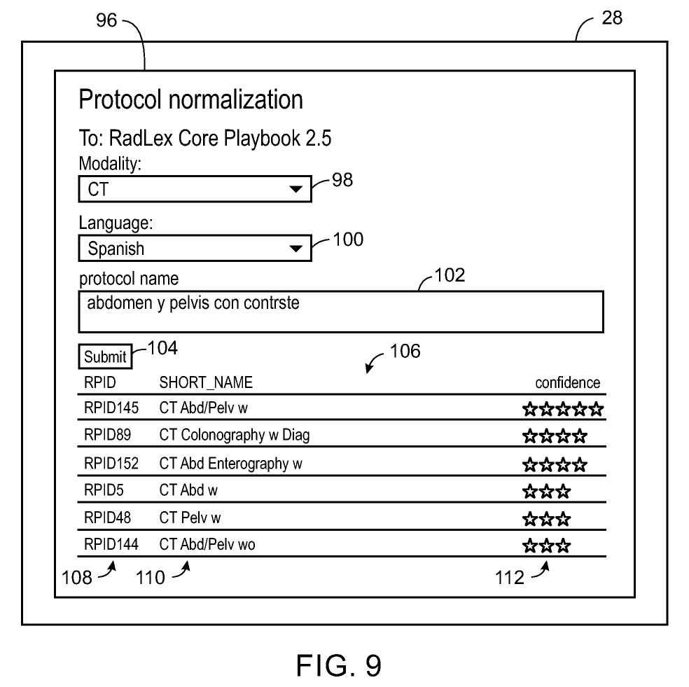

FIG. 9 is an example of a graphical user interface 96 on a display 28 for output of a lightweight text classification model. The graphical user interface 96 may vary from that depicted in FIG. 9. The graphical user interface 96 includes a target imaging modality dropdown field 98 for selecting a target imaging modality. As depicted, the selected target imaging modality was computed tomography (CT). The graphical user interface 96 also includes a language dropdown field 100 for selecting a language for the inputted medical imaging protocol name. As depicted, the selected language was Spanish. In certain embodiments, the language of the medical imaging protocol name that is received is detected. The graphical user interface 96 also includes a field 102 for entering the medical imaging protocol name. As depicted, the medical imaging protocol name provided is in Spanish and also includes a typo. The graphical user interface 96 further includes a button 104 submit the target imaging modality, the language of the inputted medical imaging protocol name, and the medical imaging protocol name. A bottom portion of the graphical user interface includes the results 106 from submitting the target imaging modality, the language of the inputted medical imaging protocol name, and the medical imaging protocol name. In particular, the results 106 is a list of the most probable protocol codes 108 (in the medical standard RadLex) based on the medical imaging protocol name. Each respective protocol code 108 is associated with a short name 110 (i.e., SHORT_NAME) with abbreviations in English. Each respective protocol code 108 is also associated with a confidence score 112. As depicted, a number of symbols (e.g., stars) represent the confidence score the higher number of stars being associated with a higher confidence score. In certain embodiments, the confidence score may be a numerical score. The embodiment in FIG. 9 demonstrates the multi-lingual aspect as the RadLex standard documentation is written in English only. Also, the embodiment in FIG. 9 demonstrates the tolerance for typos as typos occur frequently in protocol names.

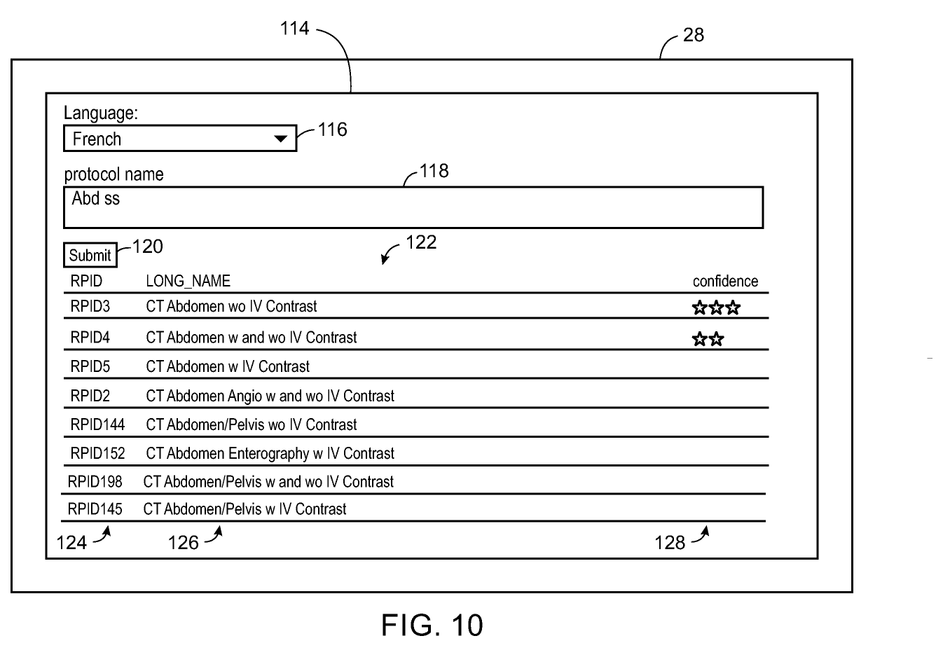

FIG. 10 is an example of a graphical user interface 114 on a display 28 for output of a lightweight text classification model. The graphical user interface 114 may vary from that depicted in FIG. 10. The graphical user interface 114 includes a language dropdown field 116 for selecting a language of the inputted medical imaging protocol name. As depicted, the selected language was French. In certain embodiments, the language of the medical imaging protocol name that is received is detected. The graphical user interface 114 also includes a field 118 for entering the medical imaging protocol name. As depicted, the medical imaging protocol name provided is in French. The graphical user interface 114 further includes a button 120 submit the target imaging modality, the language of the inputted medical imaging protocol name, and the medical imaging protocol name. A bottom portion of the graphical user interface includes the results 122 from submitting the target imaging modality, the language of the inputted medical imaging protocol name, and the medical imaging protocol name. In particular, the results 122 is a list of the most probable protocol codes 124 (in the medical standard RadLex) based on the medical imaging protocol name. Each respective protocol code 124 is associated with a name 126 (i.e., LONG_NAME) with abbreviations in English. Each respective protocol code 124 is also associated with a confidence score 128. As depicted, a number of symbols (e.g., stars) represent the confidence score the higher number of stars being associated with a higher confidence score. In certain embodiments, the confidence score may be a numerical score. “Abd ss” is a common protocol name in France. However, “ss” is a French abbreviation for “sans” (i.e., “without”) and “sans” itself is an abbreviation for “sans contraste (i.e., “without contrast”).

Technical effects of the disclosed embodiments include enabling the best large language models to be utilized without any privacy issues. In addition, technical effects of the disclosed embodiments include supporting a large number of languages with limited human involvement as the needed medical knowledge (and associated variations in variation) is extracted from a large model with no privacy issues. Technical effects of the disclosed embodiments include enabling data to be generated in various languages from an English standard without explicit translation. Technical effects of the disclosed embodiments include reducing the costs typically associated with manually mapping. Technical effects of the disclosed embodiments include providing a classifier that is nearly as accurate as using a state of the art LLM. Technical effects of the disclosed embodiments include providing a much faster and cheaper option than using a large LLM as the classification models are comparatively light and have an inference time of milliseconds. Technical effects of the disclosed embodiments include enabling the training set to be parameterized to needed cases (e.g., abbreviations).

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function]…” or “step for [perform]ing [a function]…”, it is intended that such elements are to be interpreted under 35 U.S.C. 112(f). However, for any claims containing elements designated in any other manner, it is intended that such elements are not to be interpreted under 35 U.S.C. 112(f).

The disclosure also provides support for computer-implemented method for medical imaging protocol name standardization, comprising: generating, via a processing system comprising one or more processors, a synthetic training dataset from medical standards in public documentation utilizing knowledge elicitation (e.g., automatic knowledge elicitation), wherein the synthetic training dataset comprises, for a given language and a given imaging modality, a plurality of combinations of possible medical imaging protocol names for respective standard protocol codes of a plurality of standard protocol codes for each standard medical imaging protocol; generating, via the processing system, a lightweight text classification model from the synthetic training dataset utilizing machine learning; and utilizing, via the processing system, the lightweight text classification model to receive a medical imaging protocol name and to output a list of most probable protocol codes based on the medical imaging protocol name. In a first example of the method, the method further comprises obtaining, via the processing system, a respective block of information pertaining to each standard protocol code of the plurality of standard protocol codes from the public documentation, wherein the public documentation is in the English language. In a second example of the method, optionally including the first example, generating the synthetic training dataset comprises, for each respective block of information for each standard protocol code, generating with a large language model, an expanded and explicit text description in the English language for the standard medical imaging protocol. In a third example of the method, optionally including one or both of the first and second examples, generating the synthetic training dataset comprises, from each expanded and explicit text description, generating with a multilingual medical large model a set of medical imaging protocol names in the given language with multiple variations and multiple abbreviations. In a fourth example of the method, optionally including one or more or each of the first through third examples, the given language is also the English language. In a fifth example, optionally including one or more or each of the first through fourth examples, the given language is different from the given language. In a sixth example, optionally including one or more or each of the first through fifth examples, generating the synthetic training dataset comprises generating multiple sets of medical imaging protocol names in the given language with multiple variations and multiple abbreviations. In a seventh example, optionally including the one or more or each of the first through sixth examples, the method further comprises generating the synthetic training dataset comprises attaching each medical imaging protocol name of the set of imaging protocol names to a corresponding standard protocol code from which it originated to define source-target lines of the synthetic training dataset for the given language. In an eighth example, optionally including the one or more or each of the first through the seventh examples, the synthetic training dataset is generated for multiple different languages for the given imaging modality. In a ninth example, optionally including the one or more each of the first through the eighth examples, the synthetic training dataset is generated for multiple different languages for multiple different imaging modalities. In a tenth example, optionally including the one or more each of the first through the ninth examples, the method further comprises generating, via the processing system, respective lightweight text classification models for different combinations of languages and imaging modalities from the synthetic training dataset utilizing machine learning; detecting, via the processing system, a language of the medical imaging protocol name that is received; and selecting, via processing system, a corresponding lightweight text classification model from the respective lightweight text classification models to utilize based on the language that is detected. In an eleventh example, optionally including the one or more each of the first through tenth examples, the lightweight text classification model utilizes logistic regression in determining the list of most probable protocol codes based on the medical imaging protocol name. In a twelfth example, optionally including the one or more each of the first through eleventh examples, the lightweight text classification model determines a most probable protocol based on the medical imaging protocol name. In a thirteenth example, optionally including the one or more each of the first through twelfth examples, the lightweight text classification model calculates and outputs a respective confidence score for each protocol code in the list of most probable protocol codes.

The disclosure also provides support for a system, comprising: a memory encoding processor-executable routines; and a processing system comprising one or more processors and configured to access the memory and to execute the processor-executable routines, wherein the processor-executable routines, when executed by the processing system, cause the processing system to: generate a synthetic training dataset from medical standards in public documentation utilizing knowledge elicitation, wherein the synthetic training dataset comprises, for a given language and a given imaging modality, a plurality of combinations of possible medical imaging protocol names for respective standard protocol codes of a plurality of standard protocol codes for each standard medical imaging protocol; generate a lightweight text classification model from the synthetic training dataset utilizing machine learning; and utilize the lightweight text classification model to receive a medical imaging protocol name and to output a list of most probable protocol codes based on the medical imaging protocol name. In a first example of the system, the processor-executable routines, when executed by the processing system, cause the processing system to obtain a respective block of information pertaining to each standard protocol code of the plurality of standard protocol codes from the public documentation, wherein the public documentation is in the English language. In a second example of the system, optionally including the first example, generating the synthetic training dataset comprises, for each respective block of information for each standard protocol code, generating with a large language model, an expanded and explicit text description in the English language for the standard medical imaging protocol. In a third example of the system, optionally including one or both of the first and second examples, generating the synthetic training dataset comprises, from each expanded and explicit text description, generating with a multilingual medical large language model a set of medical imaging protocol names in the given language with multiple variations and multiple abbreviations. In a fourth example of the system, optionally including one or more or each of the first through third examples, generating the synthetic training dataset comprises generating multiple sets of medical imaging protocol names in the given language with multiple variations and multiple abbreviations.

The disclosure also provides support for a non-transitory computer-readable medium, the non-transitory computer-readable medium comprising processor-executable code that when executed by a processing system comprising one or more processors, causes the processing system to: generate a synthetic training dataset from medical standards in public documentation utilizing knowledge elicitation, wherein the synthetic training dataset comprises, for a given language and a given imaging modality, a plurality of combinations of possible medical imaging protocol names for respective standard protocol codes of a plurality of standard protocol codes for each standard medical imaging protocol; generate a lightweight text classification model from the synthetic training dataset utilizing machine learning; and utilize the lightweight text classification model to receive a medical imaging protocol name and to output a list of most probable protocol codes based on the medical imaging protocol name.

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system, comprising:
a memory encoding processor-executable routines; and
a processing system comprising one or more processors and configured to access the memory and to execute the processor-executable routines, wherein the processor-executable routines, when executed by the processing system, cause the processing system to:
generate a synthetic training dataset from medical standards in public documentation utilizing knowledge elicitation, wherein the synthetic training dataset comprises, for a given language and a given imaging modality, a plurality of combinations of possible medical imaging protocol names for respective standard protocol codes of a plurality of standard protocol codes for each standard medical imaging protocol;
generate a lightweight text classification model from the synthetic training dataset utilizing machine learning; and
utilize the lightweight text classification model to receive a medical imaging protocol name and to output a list of most probable protocol codes based on the medical imaging protocol name.

2. The system of claim 1, wherein the processor-executable routines, when executed by the processing system, cause the processing system to obtain a respective block of information pertaining to each standard protocol code of the plurality of standard protocol codes from the public documentation, wherein the public documentation is in the English language.

3. The system of claim 2, wherein generating the synthetic training dataset comprises, for each respective block of information for each standard protocol code, generating with a large language model, an expanded and explicit text description in the English language for the standard medical imaging protocol.

4. The system of claim 3, wherein generating the synthetic training dataset comprises, from each expanded and explicit text description, generating with a multilingual medical large language model a set of medical imaging protocol names in the given language with multiple variations and multiple abbreviations.

5. The system of claim 4, wherein generating the synthetic training dataset comprises generating multiple sets of medical imaging protocol names in the given language with multiple variations and multiple abbreviations.

6. A computer-implemented method for medical imaging protocol name standardization, comprising:
generating, via a processing system comprising one or more processors, a synthetic training dataset from medical standards in public documentation utilizing knowledge elicitation, wherein the synthetic training dataset comprises, for a given language and a given imaging modality, a plurality of combinations of possible medical imaging protocol names for respective standard protocol codes of a plurality of standard protocol codes for each standard medical imaging protocol;
generating, via the processing system, a lightweight text classification model from the synthetic training dataset utilizing machine learning; and
utilizing, via the processing system, the lightweight text classification model to receive a medical imaging protocol name and to output a list of most probable protocol codes based on the medical imaging protocol name.

7. The computer-implemented method of claim 6, further comprising obtaining, via the processing system, a respective block of information pertaining to each standard protocol code of the plurality of standard protocol codes from the public documentation, wherein the public documentation is in the English language.

8. The computer-implemented method of claim 7, wherein generating the synthetic training dataset comprises, for each respective block of information for each standard protocol code, generating with a large language model, an expanded and explicit text description in the English language for the standard medical imaging protocol.

9. The computer-implemented method of claim 8, wherein generating the synthetic training dataset comprises, from each expanded and explicit text description, generating with a multilingual medical large language model a set of medical imaging protocol names in the given language with multiple variations and multiple abbreviations.

10. The computer-implemented method of claim 9, wherein the given language is also the English language.

11. The computer-implemented method of claim 9, wherein the given language is different from the given language.

12. The computer-implemented method of claim 9, wherein generating the synthetic training dataset comprises generating multiple sets of medical imaging protocol names in the given language with multiple variations and multiple abbreviations.

13. The computer-implemented method of claim 9, wherein generating the synthetic training dataset comprises attaching each medical imaging protocol name of the set of imaging protocol names to a corresponding standard protocol code from which it originated to define source-target lines of the synthetic training dataset for the given language.

14. The computer-implemented method of claim 6, wherein the synthetic training dataset is generated for multiple different languages for the given imaging modality.

15. The computer-implemented method of claim 6, wherein the synthetic training dataset is generated for multiple different languages for multiple different imaging modalities.

16. The computer-implemented method of claim 15, further comprising:
generating, via the processing system, respective lightweight text classification models for different combinations of languages and imaging modalities from the synthetic training dataset utilizing machine learning;
detecting, via the processing system, a language of the medical imaging protocol name that is received; and
selecting, via processing system, a corresponding lightweight text classification model from the respective lightweight text classification models to utilize based on the language that is detected.

17. The computer-implemented method of claim 6, wherein the lightweight text classification model utilizes logistic regression in determining the list of most probable protocol codes based on the medical imaging protocol name.

18. The computer-implemented method of claim 6, wherein the lightweight text classification model determines a most probable protocol based on the medical imaging protocol name.

19. The computer-implemented method of claim 6, wherein the lightweight text classification model calculates and outputs a respective confidence score for each protocol code in the list of most probable protocol codes.

20. A non-transitory computer-readable medium, the non-transitory computer-readable medium comprising processor-executable code that when executed by a processing system comprising one or more processors, causes the processing system to:
generate a synthetic training dataset from medical standards in public documentation utilizing knowledge elicitation, wherein the synthetic training dataset comprises, for a given language and a given imaging modality, a plurality of combinations of possible medical imaging protocol names for respective standard protocol codes of a plurality of standard protocol codes for each standard medical imaging protocol;
generate a lightweight text classification model from the synthetic training dataset utilizing machine learning; and
utilize the lightweight text classification model to receive a medical imaging protocol name and to output a list of most probable protocol codes based on the medical imaging protocol name.
